(19) United States
(12) Patent Application Publication
(10) Pub. No.: US 2025/0325549 A1
(43) Pub. Date: Oct. 23, 2025

(54) ORAL TABLET COMPOSITION OF RUXOLITINIB AND METHOD FOR PREPARING SAME

(71) Applicant: SAMYANG HOLDINGS CORPORATION, Seoul (KR)

(72) Inventors: Nam Hyuck KIM, Uiwang-si (KR); Sang Yeob PARK, Yongin-si (KR); Dong Sik KIM, Suwon-si (KR)

(73) Assignee: SAMYANG HOLDINGS CORPORATION, Seoul (KR)

(21) Appl. No.: 18/684,468

(22) PCT Filed: Aug. 17, 2022
(86) PCT No.: PCT/KR2022/012298

§ 371 Date: Jul. 30, 2024

(30) Foreign Application Priority Data
Aug. 18, 2021 (KR) 10-2021-0108537

(51) Int. Cl. A61K 31/519; A61K 9/20; A61K 9/28

(52) U.S. Cl. CPC A61K 31/519; A61K 9/2013; A61K 9/2018; A61K 9/2031; A61K 9/2054; A61K 9/2095; A61K 9/2893

(57) ABSTRACT

The present invention relates to a sustained-release formulation of ruxolitinib or a pharmaceutically acceptable salt thereof which is useful for the treatment of Janus kinase-associated diseases such as myeloproliferative disorders, and a method for preparing same.

### TECHNICAL FIELD

The present invention relates to a sustained-release formulation of Ruxolitinib or a pharmaceutically acceptable salt thereof which is useful for the treatment of Janus kinase-associated disease such as myeloproliferative disease, and a method for preparing the same.

### BACKGROUND ART

Ruxolitinib ((3R)-3-cyclopentyl-3-[4-(7H-pyrrolo[2,3-d]pyrimidin-4-yl)pyrazol-1-yl]propanenitrile) is the first FDA-approved Janus kinase (JAK) inhibitor and the first drug approved for the treatment of myelofibrosis. It is marketed in Korea under the product name Jakavi® and is administered twice a day.

Ruxolitinib is a BCS class I molecule with rapid oral absorption and a short half-life of approximately 3 hours [Shi et. al.., J. Clin. Pharmacol. 2012 June; 52 (6): 809-18. Epub 2011 May 20]. These characteristics cause the highest peak/lowest plasma concentration ratio in human subjects, resulting in multiple daily doses for optimal treatment, and potentially contributing to problems with patient compliance and unwanted side effects. Ruxolitinib therapy is commonly associated with the side effects of thrombocytopenia (low platelet count) and anemia (low hemoglobin). Thrombocytopenia is dose-dependent and dose-limiting toxic effects are considered. Accordingly, there is a need for novel and improved formulation of Ruxolitinib which alleviates side effects in patients while still achieving therapeutic efficacy, and also facilitates administration of the drug, such as by reducing the number of doses required to achieve therapeutic effect.

Korean Laid-open Patent Publication No. 10-2015-0085833 discloses a sustained-release formulation of Ruxolitinib intended for once-daily administration, but the formulation disclosed in this document contains hydroxypropyl methylcellulose which can cause rapid initial release of the drug, and due to the physicochemical properties of Ruxolitinib having higher solubility at low pH, initial release control upon exposure to gastric acid, etc. is not efficient.

### CONTENTS OF THE INVENTION

### Problems to be Solved

The present invention is to resolve the problem of the prior art as explained above, and the purpose of the present invention is to provide a sustained-release formulation of Ruxolitinib which can resolve the rapid initial release problem of the existing Ruxolitinib formulation and exhibit more improved drug release pattern and improved drug absorption rate, and a method for preparing the same.

### TECHNICAL MEANS

In order to achieve the above purpose, one aspect of the present invention provides an oral tablet formulation comprising Ruxolitinib as an active ingredient; and polyethylene oxide as a sustained-release agent.

In an embodiment, the oral tablet formulation of the present invention further comprises a sustained-release aid.

In an embodiment, the sustained-release aid is a hydrophobic material or a hydrophilic material.

In an embodiment, the hydrophobic sustained-release aid may be selected from glyceryl behenate, ethylcellulose, ammonium methacrylate copolymer, shellac, hydroxypropylmethylcellulose phthalate (HPMC-P), waxes, gums, and combinations thereof.

In an embodiment, the hydrophilic sustained-release aid may be selected from polyethylene glycol, povidone, hydroxypropylcellulose, sugar alcohols, and combinations thereof.

In an embodiment, the oral tablet formulation of the present invention may exhibit a dissolution pattern with the coefficient of determination, R2 of 0.93 or more obtained by linear regression model of the dissolution time-dissolution rate curve in purified water from the time for the drug dissolution rate being 0% to the time for the drug dissolution rate becoming 85% or more, based on uncoated tablet.

The other aspect of the present invention provides a method for preparing an oral tablet formulation, the method comprising: (1) a step of preparing a mixture comprising Ruxolitinib as an active ingredient and polyethylene oxide as a sustained-release agent; and (2) a step of tableting the mixture.

In an embodiment, the mixture tableted in step (2) of the method for preparing an oral tablet formulation of the present invention further comprises a sustained-release aid as explained above.

### Effect of the Invention

The oral tablet formulation of Ruxolitinib provided according to the present invention exhibits the characteristics of a sustained-release formulation without rapid initial release of the drug, and minimizes the change in the drug release pattern due to pH change, thereby efficiently maintaining the blood concentration of the drug in the body to the effective level or higher. Therefore, according to the present invention, it is possible to obtain a sustained-release formulation of Ruxolitinib which can resolve the rapid initial release problem of the existing Ruxolitinib formulation and exhibit more improved drug release pattern and improved drug absorption rate.

### PREFERRED MODE FOR CARRYING OUT THE INVENTION

### Definition of Terms

Unless explicitly described otherwise, some terms used in the entire specification of the present invention can be defined as follows.

In the entire specification of the present invention, unless mentioned especially otherwise, “comprising” or “containing” refers to comprising a certain constitutional element (or constitutional component) without special limitation, and it is not interpreted as excluding addition of other constitutional element (or constitutional component).

The present invention is explained in more detail below.

The oral tablet formulation of Ruxolitinib according to the present invention comprises Ruxolitinib as an active ingredient; and polyethylene oxide as a sustained-release agent.

In the present invention, the “Ruxolitinib” may be the free base of Ruxolitinib (a base drug without a separate salt), or a pharmaceutically acceptable salt thereof (e.g., a phosphate salt), an isomer thereof, or a mixture thereof. Also, in each case, it may form one of various hydrates, and in each case, it may form one of various crystal forms. For example, it may be Ruxolitinib anhydrate, one of various hydrates of Ruxolitinib such as hemihydrate, monohydrate, dihydrate, trihydrate, etc. or one of various solvates thereof, or a mixture thereof.

In an embodiment, the “Ruxolitinib or a pharmaceutically acceptable salt thereof” may be Ruxolitinib phosphate salt.

In the present invention, the polyethylene oxide used as a sustained-release agent is not especially limited as long as it can control the release of the drug from the formulation. In an embodiment, as such polyethylene oxide (hereinafter, it may be abbreviated as PEO), for example, Polyox WSR-303, Polyox WSR-301, Polyox WSR N-60K, Polyox WSR-205, Polyox WSR-N80 or a combination thereof may be used, but it is not limited thereto. In the present invention, two or more PEOs different in molecular weight, grade, etc. may be combined and used.

In an embodiment, the viscosity average molecular weight (g/mol) of PEO (when two or more PEOs are used in combination, it refers to the viscosity average molecular weight of the combination) may be, for example, 50,000 or more, 100,000 or more, 150,000 or more, or 200,000 or more, and it also may be 7,000,000 or less, 6,00,000 or less, 5,000,000 or less, 4,000,000 or less, 3,000,000 or less, or 2,000,000 or less, and more concretely, it may be 100,000 to 4,000,000 g/mol, 150,000 to 3,000,000 g/mol, or 200,000 to 2,000,000 g/mol, but it is not limited thereto. If the viscosity average molecular weight of PEO is too less than the above level, the drug is released too quickly and so there may be no meaning as a sustained-release formulation, and to the contrary, if it is too greater than the above level, the formulation shows high blood concentration even 24 hours after in vivo administration, and upon repeated administration, accumulation of drug concentration in the blood may occur.

In an embodiment, the viscosity of PEO (when two or more PEOs are used in combination, it refers to the viscosity of the combination) may be, based on 1 to 5% (wt/wt) aqueous solution at 25° C., for example, 1000 cP or more, 1100 cP or more, 1200 cP or more, 1300 cP or more, 1400 cP or more, or 1500 cP or more, and it also may be 10000 cP or less, 9000 cP or less, 8000 cP or less, 7000 cP or less, or 6000 cP or less. For example, the viscosity of PEO may be 1000 cP to 6000 cP based on 1% (wt/wt) aqueous solution at 25° C., or 1500 cP to 7000 cP based on 2% (wt/wt) aqueous solution at 25° C., or 4000 cP to 10000 cP based on 5% (wt/wt) aqueous solution at 25° C., but it is not limited thereto. If the viscosity of PEO is too less than the above level, the drug is released too quickly and so there may be no meaning as a sustained-release formulation, and to the contrary, if it is too greater than the above level, the formulation shows high blood concentration even 24 hours after in vivo administration, and upon repeated administration, accumulation of drug concentration in the blood may occur.

In an embodiment, the amount of PEO in the oral tablet formulation of the present invention may be, based on 1 part by weight of Ruxolitinib, 1 part by weight or more, 5 parts by weight or more, 10 parts by weight or more, 20 parts by weight or more, 30 parts by weight or more, or 40 parts by weight or more, and it also may be 90 parts by weight or less, 80 parts by weight or less, or 70 parts by weight or less, and more concretely, it may be 10 to 90 parts by weight, 20 to 80 parts by weight, or 40 to 70 parts by weight, but it is not limited thereto. If the amount of PEO in the oral tablet formulation of the present invention is too less than the above level, the drug is released too quickly and so there may be no meaning as a sustained-release formulation, and to the contrary, if it is too greater than the above level, the proportion of viscous polymer increases, and thus tableting may be impossible and the drug may be excreted from the body without being sufficiently released therein.

Since the PEO shows a phenomenon of dissolving and gelling immediately upon contact with an organic solvent, for example, alcohol, especially ethanol, wet granulation using such a solvent and subsequent drying, sieving and tableting processes cannot be applied.

Therefore, in an embodiment, the oral tablet formulation of the present invention does not comprise an organic solvent (for example, alcohol such as ethanol). That is, in an embodiment, the oral tablet formulation of the present invention is prepared by a dry process not using an organic solvent (for example, alcohol such as ethanol).

In an embodiment, the oral tablet formulation of the present invention further comprises a sustained-release aid.

In an embodiment, the sustained-release aid may be a hydrophobic material with resistance to moisture penetration, or a hydrophilic material with assistance for moisture penetration and function of viscosity assistance.

In an embodiment, the hydrophobic sustained-release aid may be selected from glyceryl behenate, ethylcellulose, ammonium methacrylate copolymer, shellac, hydroxypropylmethylcellulose phthalate (HPMC-P), waxes, gums, and combinations thereof.

The glyceryl behenate, if comprised in the oral tablet formulation of the present invention, serves as an appropriate lubricant during the dry granulation process and compression molding process, and also maintains the shape of the tablet and delays drug release due to its strong hydrophobic characteristics.

In an embodiment, the ammonium methacrylate copolymer may be poly(ethyl acrylate/methyl methacrylate/trimethylammonium chloride methacrylate) (e.g., Eudragit® RL or Eudragit® RS, Evonik), but it is not limited thereto.

In an embodiment, the wax may be carnauba wax, beeswax, microcrystalline wax, or combination thereof, and the gum may be guar gum, locust bean gum, tragacantha, carrageenan, acacia gum, gum arabic, gellan gum, xanthan gum, or combination thereof, but not limited thereto.

In an embodiment, the hydrophilic sustained-release aid may be selected from polyethylene glycol, povidone, hydroxypropylcellulose, sugar alcohols, and combinations thereof.

In an embodiment, the number average molecular weight (g/mol) of the polyethylene glycol (e.g., product name Macrogol) may be 1,000 to 10,000 g/mol, but it is not limited thereto.

In an embodiment, the sugar alcohol may be sorbitol, maltitol, xylitol, erythritol or combination thereof, but it is not limited thereto.

In a preferable embodiment, the hydrophobic sustained-release aid may be glyceryl behenate, and he hydrophilic sustained-release aid may be polyethylene glycol.

In an embodiment, the amount of the sustained-release aid in the oral tablet formulation of the present invention may be, based on 1 part by weight of Ruxolitinib, 0.05 part by weight or more, 0.1 part by weight or more, 0.12 part by weight or more, or 0.15 part by weight or more, and it also may be 5 parts by weight or less, 4 parts by weight or less, 3 parts by weight or less, or 2 parts by weight or less, but it is not limited thereto. If the amount of the sustained-release aid in the oral tablet formulation of the present invention is too less than the above level, there may be problems such as failure in dry granulation process and compression molding process and collapse of tablet shape after gelation, and to the contrary, if it is too greater than the above level, there may be problems such as failure in dry granulation process and compression molding process due to binding strength reduction. If the sustained-release aid (e.g., glyceryl behenate) functions as a lubricant simultaneously, its amount may be, based on the total weight of the tablet, 5 w/w % to 20 w/w %, 10 w/w % to 20 w/w %, or 10 w/w % to 15 w/w %.

In addition to the above explained ingredients, the oral tablet formulation of the present invention may further comprise one or more of pharmaceutically acceptable carriers or additives.

In an embodiment, the oral tablet formulation of the present invention may further comprise a diluent.

In an embodiment, the diluent may be selected from the group consisting of sugar, sugar alcohol, cellulose, starch, inorganic salt and mixtures thereof, and more concretely, it may be selected from the group consisting of lactose (anhydrate or hydrate, e.g., monohydrate), cellulose powder, microcrystalline cellulose, silicified microcrystalline cellulose, starch, pregelatinized starch, calcium carbonate, cyclodextrin, calcium sulfate, calcium silicate, magnesium carbonate, dicalcium phosphate, tricalcium phosphate, magnesium trisilicate, potassium chloride, sodium chloride, dibasic calcium phosphate dihydrate, tribasic calcium phosphate, kaolin, magnesium carbonate, magnesium oxide, mannitol, maltitol, sorbitol, xylitol, lactose, dextrose, maltose, sucrose, glucose, fructose, maltodextrin, dextrates, dextrin and mixtures thereof. Preferably, lactose or mannitol may be selected from the above.

In an embodiment, there may be some cases where the diluent may also function as a binder.

In an embodiment, if the diluent is used in the oral tablet formulation of the present invention, its amount of use may be, based on 1 part by weight of Ruxolitinib, 0.5 part by weight or more, 1 part by weight or more, or 1.5 parts by weight or more, and it also may be 400 parts by weight or less, 200 parts by weight or less, 100 parts by weight or less, 50 parts by weight or less, or 20 parts by weight or less, but it is not limited thereto. The amount of the diluent within the above range is suitable for tablet preparation.

In an embodiment, the oral tablet formulation of the present invention may further comprise a lubricant.

In an embodiment, the lubricant may be selected from the group consisting of soluble lubricant, insoluble lubricant and mixtures thereof, and more concretely, it may be selected from the group consisting of magnesium stearate, fumaric acid, stearic acid, calcium stearate, sodium stearyl fumarate, sucrose fatty acid ester, starch, talc, colloidal silica, magnesium oxide, magnesium carbonate, glyceryl monostearate, silicon dioxide, calcium silicate, magnesium silicate, hardened vegetable oil, light liquid paraffin, polyethylene glycol, sodium lauryl sulfate, magnesium lauryl sulfate, sodium benzoate, polyoxyethylene monostearate, glyceryl triacetate, sucrose monolaurate and mixtures thereof. Among them, preferably magnesium stearate, stearic acid or colloidal silica may be selected, and more preferably magnesium stearate may be selected.

In an embodiment, if the lubricant is used in the oral tablet formulation of the present invention, its amount of use may be, based on 1 part by weight of Ruxolitinib, 0.005 part by weight or more, 0.01 part by weight or more, or 0.05 part by weight or more, and it also may be 10 parts by weight or less, 5 parts by weight or less, or 1 part by weight or less, but it is not limited thereto. The amount of the lubricant within the above range is suitable in terms of tableting stability and productivity of the tablet.

In the oral tablet formulation of the present invention, the PEO described above can act as a binder in the tableting process. Accordingly, in an embodiment the oral tablet formulation of the present invention may not comprise a binder. However, the possibility of using a binder in the oral tablet formulation of the present invention is not completely excluded, and thus in other embodiment, the oral tablet formulation of the present invention may further comprise a binder, and its kind and amount may be properly selected within the scope that can achieve the purpose of the present invention.

The oral tablet formulation of the present invention may further comprise a coating layer.

In an embodiment, a coating base material that forms the coating layer may be selected from the group consisting of polyvinylpyrrolidone, hydroxypropylmethylcellulose, carboxymethylcellulose and its salt, ethylcellulose, methylcellulose, hydroxyethylcellulose, ethylhydroxyethylcellulose, hydroxypropylcellulose, low-substituted hydroxypropylcellulose, polyvinyl alcohol, macrogol polyvinyl alcohol graft copolymer, polymer of acrylic acid and its salt, polymethacrylate, poly(butyl methacrylate, 2-dimethylaminoethyl methacrylate, methyl methacrylate) copolymer, vinylpyrrolidone-vinyl acetate copolymer, gelatin, guar gum, partially hydrolyzed starch, alginate, xanthan and mixtures thereof.

In an embodiment, if the oral tablet formulation of the present invention further comprises a coating layer, its amount may be, based on 100 parts by weight of the tablet before coating (uncoated tablet), 1 part by weight or more, 2 parts by weight or more, 3 parts by weight or more, 4 parts by weight or more, 5 parts by weight or more, or 6 parts by weight or more, and it also may be 30 parts by weight or less, 25 parts by weight or less, 20 parts by weight or less, or 15 parts by weight or less, but it is not limited thereto. If the amount of the coating layer is less than the above range, the entire uncoated tablet may not be sufficiently coated, and to the contrary, if it is greater than the above range, delay in dissolution rate may occur.

The purpose of the coating is to improve the stability of the uncoated tablet and prevent the drug from disappearing. Therefore, if necessary, a second coating may be applied after the first coating, and the thickness of the coating may also be appropriately selected. However, since dissolution of the drug should not be delayed due to the coating as described above, it is necessary to select an appropriate range. The other ingredients and methods required for coating may be appropriately adopted by a skilled artisan.

In an embodiment, the hardness of the tablet (uncoated tablet) before coating of the oral tablet formulation of the present invention may be 10N to 400N. More concretely, the minimum average hardness of the uncoated tablet of the oral tablet formulation of the present invention may be 10N, 20N, 30N or 50N, and the maximum average hardness thereof may be 400N, 300N, 200N or 150N. If the hardness of the uncoated tablet is higher than the above level, problems may occur in the process due to excessive tableting pressure, and to the contrary, if it is lower than the above level, the tablet becomes weak and may be broken during coating, transportation, storage, packaging, or administration.

In an embodiment, the oral tablet formulation of the present invention may have a dissolution rate of 85% or less, 80% or less, 75% or less, 70% or less, or 65% or less within 16 hours or 12 hours in purified water at 37° C. under 50 rpm condition.

Also, in an embodiment, the oral tablet formulation of the present invention may have a dissolution rate of 70% or less, 60% or less, or 50% or less in 8 hours or 5 hours in 0.1N HCl aqueous solution at 37° C. under 50 rpm condition.

In an embodiment, the oral tablet formulation of the present invention may exhibit a dissolution pattern with the coefficient of determination, R2 of 0.93 or more obtained by linear regression model of the dissolution time-dissolution rate curve in purified water from the time for the drug dissolution rate being 0% to the time for the drug dissolution rate becoming 85% or more, based on uncoated tablet. More concretely, the coefficient of determination, R2 may be 0.95 or more, 0.97 or more, 0.99 or more, or 0.995 or more (the maximum value of R2 is 1). The dissolution pattern may be determined, for example, by Method 2 (paddle, 50 rpm) of the Korean Pharmacopoeia Dissolution Test Method.

The coefficient of determination, R2 represents the degree of difference between the dissolution time (X)-dissolution rate (Y) trend line derived through linear regression model from the dissolution time-dissolution rate curve and the actually measured value, to find out the linearity of the drug release from the time for the drug dissolution rate being 0% to the time for the drug dissolution rate becoming 85% or more, and it is an indicator showing whether the linear regression model is well fitted linearly. It can be said that R2 becomes closer to 1, all response variables fit more with predicted variables. That is, as the R2 value of the first-order equation by the trend line becomes closer to 1, it is more possible to control as zero-order dissolution, and this means that the drug is released at the same rate in the body so that the effective drug concentration in blood is maintained. The coefficient of determination, R2 is calculated according to the following equation:

$$\begin{matrix}{R}^{2} = \frac{SSE}{SST} = 1 - \frac{SSR}{SST} \\ SST = {\sum }_{i = 1}^{n} {( {y}_{i} - \overline{y} )}^{2} \\ SSE = {\sum }_{i = 1}^{n} {( {\hat{y}}_{i} - \overline{y} )}^{2} \\ SSR = {\sum }_{i = 1}^{n} {( {y}_{i} - \hat{{y}_{i}} )}^{2}\end{matrix}$$

In an embodiment, the shape of the oral tablet formulation of the present invention may be various such as rectangular, oval, diamond, circular, polygonal (e.g., triangular, square, pentagonal, hexagonal, etc.). The shape of the tablet may be determined by comprehensively judging convenience of taking it for the patient, easiness in punch manufacturing and management, easiness relating to manufacturing such as tableting, coating, packaging, handling, etc., controllability of dissolution pattern, easiness in controlling variables relating to properties such as hardness, friability, disintegration, etc. of the tablet. In addition, the appropriate shape may be selected according to each dose.

In an embodiment, particularly even for the maximum dose, the total weight of the tablet (uncoated tablet) before coating of the oral tablet formulation of the present invention, as the average weight of the tablet, is preferably not more than 1100 mg. It may be more preferably not more than 880 mg, even more preferably not more than 660 mg, even more preferably not more than 550 mg, even more preferably not more than 440 mg, and most preferably not more than 350 mg.

In an embodiment, the oral tablet formulation of the present invention is useful for the treatment of Janus kinase-associated disease, more concretely, myeloproliferative disease.

The other aspect of the present invention provides a method for preparing an oral tablet formulation, the method comprising: (1) a step of preparing a mixture comprising Ruxolitinib as an active ingredient and polyethylene oxide as a sustained-release agent; and (2) a step of tableting the mixture.

According to an embodiment, the mixture tableted in step (2) of the method for preparing an oral tablet formulation of the present invention further comprises a sustained-release aid.

In the method for preparing an oral tablet formulation, Ruxolitinib as an active ingredient, polyethylene oxide as a sustained-release agent, and sustained-release aid as a further ingredient are the same as explained above.

In an embodiment, the mixture tableted in step (2) may further comprise a diluent, and the diluent that can be used herein is the same as explained above.

In an embodiment, the mixture tableted in step (2) may further comprise a lubricant, and the lubricant that can be used herein is the same as explained above.

In an embodiment, the mixture tableted in step (2) may further comprise a binder, and the binder that can be used herein is the same as explained above.

In an embodiment, the method for preparing an oral tablet formulation of the present invention may further comprise (3) a step of coating the surface of the tablet (uncoated tablet) obtained as a result of tableting in step (2) with a coating base material, and the coating base material that can be used herein is the same as explained above.

A general method for preparing an oral solid formulation comprises the following steps:
Step 1: Process of mixing additives other than lubricant (e.g., sustained-release agent, sustained-release aid, diluent, binder, etc.)
Step 2: Process of adding drug to the mixture of the above additives and mixing them
Step 3: Process of granulating the mixture of the drug and additives
Step 4: Process of mixing the granules and lubricant
Step 5: Process of tableting the granules that have completed the lubrication process
Step 6: Process of coating the tablet

In addition, a process of uniformizing the particle size of the mixture through sieving may be added in each of the above steps. This process may help with the flowability and compression moldability of the mixture.

The oral tablet formulation of the present invention may be prepared by applying the above general method as is or with appropriate modification.

For example, in an embodiment, the oral tablet formulation of the present invention may be prepared in a manner that Ruxolitinib as an active ingredient, polyethylene oxide as a sustained-release agent and a diluent are sieved and mixed; then glyceryl behenate or macrogol as a sustained-release aid, and a lubricant are further added thereto and mixed; and the resulting mixture is tableted. In other embodiment, it may be prepared in a manner that Ruxolitinib, polyethylene oxide, a diluent, and glyceryl behenate or macrogol as a sustained-release aid are sieved and mixed; then a lubricant is further added thereto and mixed; and the resulting mixture is tableted. In another embodiment, it may be prepared in a manner that Ruxolitinib, polyethylene oxide, a diluent, glyceryl behenate or macrogol as a sustained-release aid, and a lubricant are sieved and mixed, and then dry compression granulation is conducted; a lubricant is further added thereto and mixed; and the resulting mixture is tableted.

In an embodiment, the preparation of the oral tablet formulation of the present invention may be carried out in the order of weighing the raw ingredients and subsequent (optional) granulation, mixing, tableting and coating. The granulation may be performed by dry granulation, wet granulation, etc.

In an embodiment, if the granulation is performed by wet granulation, the granules are obtained by preparing a binder solution, adding thereto the drug together with a diluent, etc. and mixing them, mixing the resulting mixture with the binder solution to form granules, and sieving and drying the granules. After that, the remaining ingredients are mixed through post-mixing, and then the resulting mixture is tableted. The binder solution is the same as explained above. As a bonder solvent, water, etc. may be used, but since PEO shows a phenomenon of dissolving and gelling immediately upon contact with an organic solvent, for example, alcohol, especially ethanol, such a solvent is not used.

In an embodiment, if the granulation is performed by dry granulation, the mixture of drug and diluent, etc. is compressed using a roller compactor, etc. and sieved, and after that, the remaining ingredients are mixed through post-mixing, and then the resulting mixture is tableted.

The following examples are provided below in order to facilitate understanding of the present invention. However, the following examples are only to illustrate the present invention, and the scope of the present invention is not limited thereby in any manner.

### EXAMPLES

### Polyethylene Oxides as Used

Polyox WSR 205: Viscosity average molecular weight: 600,000 g/mol, Viscosity (5% aqueous solution at 25° C.): 4500 to 8800 cP
Polyox WSR N60K: Viscosity average molecular weight: 2,000,000 g/mol, Viscosity (2% aqueous solution at 25° C.): 2000 to 4000 cP
Polyox WSR 301: Viscosity average molecular weight: 4,000,000 g/mol, Viscosity (1% aqueous solution at 25° C.): 1650 to 5500 cP
Polyox WSR N80: Viscosity average molecular weight: 200,000 g/mol, Viscosity (5% aqueous solution at 25° C.): 65 to 90 cP

### Sustained-Release Aids as Used

Glyceryl behenate
Macrogol 4000 (polyethylene glycol with number average molecular weight 4000 g/mol)

### Example 1

### Preparation of Uncoated Tablet

2.64 g of Ruxolitinib phosphate, 18.5 g of Polyox WSR 205, and 5.12 g of mannitol were sieved and mixed, and then 0.44 g of glyceryl behenate and 0.3 g of magnesium stearate were added thereto and mixed (lubrication step). The mixture was tableted with a rectangular punch based on the weight of 270.0 mg per tablet.

### Example 2

### Preparation of Uncoated Tablet

2.64 g of Ruxolitinib phosphate, 18.5 g of Polyox WSR N60K, and 5.12 g of mannitol were sieved and mixed, and then 0.44 g of glyceryl behenate and 0.3 g of magnesium stearate were added thereto and mixed (lubrication step). The mixture was tableted with a rectangular punch based on the weight of 270.0 mg per tablet.

### Example 3

### Preparation of Uncoated Tablet

2.64 g of Ruxolitinib phosphate, 18.5 g of Polyox WSR 301, and 5.12 g of mannitol were sieved and mixed, and then 0.44 g of glyceryl behenate and 0.3 g of magnesium stearate were added thereto and mixed (lubrication step). The mixture was tableted with a rectangular punch based on the weight of 270.0 mg per tablet.

### Example 4

### Preparation of Uncoated Tablet

2.64 g of Ruxolitinib phosphate, 15.0 g of Polyox WSR 205, 5.12 g of mannitol, and 4.5 g of glyceryl behenate were sieved and mixed, and then 0.3 g of magnesium stearate was added thereto and mixed (lubrication step). The mixture was tableted with a rectangular punch based on the weight of 275.6 mg per tablet.

### Comparative Example 1

### Preparation of Uncoated Tablet

2.64 g of Ruxolitinib phosphate, 4.75 g of microcrystalline cellulose, 9.16 g of lactose hydrate, 0.87 g of hydroxypropyl methylcellulose (2208 K15), and 3.46 g of hydroxypropyl methylcellulose (2208 K4) were sieved and mixed. Then, 6.0 g of water was added thereto and mixed, and the resulting mixture was dried at 50° C. to volatilize the solvent, and then sieved to obtain granules.

To the sieved granules, 0.216 g of light anhydrous silicic acid, 0.104 g of magnesium stearate, and 0.432 g of stearic acid were added and mixed (lubrication step). The mixture was tableted with a rectangular punch based on the weight of 216.4 mg per tablet.

### Comparative Example 2

### Preparation of Uncoated Tablet

2.64 g of Ruxolitinib phosphate, 4.75 g of microcrystalline cellulose, and 9.16 g of lactose hydrate were sieved and mixed. Then, a solution of 5.69 g of Eudragit RS dissolved in 10.0 g of ethanol was added thereto and mixed, and the resulting mixture was dried at 50° C. to volatilize the solvent, and then sieved to obtain granules.

To the sieved granules, 0.28 g of magnesium stearate was added and mixed (lubrication step). The mixture was tableted with a rectangular punch based on the weight of 270.0 mg per tablet.

### Comparative Example 3

### Preparation of Wet Tablet

Applied was a process wherein 2.64 g of Ruxolitinib phosphate, 18.5 g of Polyox WSR 205, and 5.12 g of mannitol were sieved and mixed, and then 6.0 g of ethanol was added thereto and mixed, and the resulting mixture was dried at 50° C. to volatilize the solvent and then sieved.

However, the Polyox showed a phenomenon of dissolving and gelling immediately upon contact with the ethanol, and the mixture had a status to which the sieving process after drying could not be applied. Thus, in Comparative Example 3, the granulation process could not be completed, and the subsequent tableting process could not be performed.

### Test Example 1: Stability Test with Severity

Each tablet prepared in Examples 1 to 4 and Comparative Examples 1 and 2 was put into HDPE bottle, and while keeping it at a 60° C. condition, the changes in the contents of Ruxolitinib and related substances in the tablet according to storage time were checked. The results are shown in Tables 1 and 2 below.
Analysis Method: HPLC Method

TABLE 1
Changes in the content of Ruxolitinib
in the tablet according to storage time

| | Initial | After 2 weeks | After 4 weeks |
|---|---|---|---|
| Comparative Example 1 | 99.9% | 99.5% | 100.5% |
| Comparative Example 2 | 101.6% | 92.7% | 87.0% |
| Example 1 | 100.9% | 100.6% | 100.9% |
| Example 2 | 99.8% | 99.6% | 99.1% |
| Example 3 | 99.9% | 101.1% | 102.2% |
| Example 4 | 99.3% | 98.8% | 101.1% |

TABLE 2
Changes in the content of related substances
in the tablet according to storage time

| | Initial | After 2 weeks | After 4 weeks |
|---|---|---|---|
| Comparative | Not detected | Not detected | Not detected |
| Example 1 | | | |
| Comparative | RRT 0.71: 0.31% | RRT 0.40: 0.07% | RRT 0.40: 0.07% |
| Example 2 | RRT 0.98: 0.18% | RRT 0.71: 0.26% | RRT 0.71: 0.33% |
| | Total: 0.49% | RRT 0.98: 0.24% | RRT 0.98: 0.34% |
| | | RRT 1.04: 0.08% | RRT 1.04: 0.32% |
| | | Total: 0.65% | Total: 1.06% |
| Example 1 | Not detected | Not detected | Not detected |
| Example 2 | Not detected | Not detected | Not detected |
| Example 3 | Not detected | Not detected | Not detected |
| Example 4 | Not detected | Not detected | Not detected |

RRT: Peak retention time of related substances/Peak retention time of main ingredient

### Test Example 2: Dissolution Test and Evaluation of Linearity of Dissolution Pattern

### 1. Dissolution Test

For each tablet prepared in Examples 1 to 4 and Comparative Example 1, a dissolution test was conducted with n=3 under the following conditions according to the paddle method of the 10th revision of the Korean Pharmacopoeia dissolution test method.

### <Dissolution Test Method>

Dissolution liquid: Purified water
Rotation speed: 50 rpm
Temperature: 37° C.
Timing of collecting dissolution liquid sample: 0.25 hour, 0.5 hour, 0.75 hour, 1 hour, 1.5 hours, 2 hours, 3 hours, 4 hours, 5 hours, 6 hours, 7 hours, 8 hours, 9 hours, 10 hours, 12 hours, 14 hours, 16 hours, 18 hours, 20 hours, 24 hours
Analysis method: HPLC method

### 2. Evaluation of Linearity of Dissolution Pattern

From the results of the dissolution test conducted for each tablet prepared in Examples 1 to 4 and Comparative Example 1, the equation between dissolution time (X) and dissolution rate (Y) was calculated as a trend line through a linear regression model of the dissolution curve from the time zero (0) to the time of about 85% dissolution, and the results are shown in Table 3 below.

TABLE 3
Linearity of dissolution pattern

| Sample | Equation | R² |
|---|---|---|
| Comparative Example 1 (uncoated tablet) | Y = 8.0X + 14.7 | 0.9265 |
| Example 1 (uncoated tablet) | Y = 11.3X + 0.2 | 0.9996 |
| Example 2 (uncoated tablet) | Y = 6.2X + 4.7 | 0.9813 |
| Example 3 (uncoated tablet) | Y = 4.6X + 7.1 | 0.9809 |
| Example 4 (uncoated tablet) | Y = 13.0X − 0.5 | 0.9993 |

Through the above test results, it can be confirmed that the release profiles of the sustained-release tablets of Examples 1 to 4 are closer to a straight line (i.e., R2 is closer to 1) as compared with Comparative Example 1.

### Example 5

### Preparation of Uncoated Tablet

2.64 g of Ruxolitinib phosphate, 15.0 g of Polyox WSR 205, 5.12 g of mannitol, 4.5 g of glyceryl behenate, and 0.1 g of magnesium stearate were sieved and mixed. The mixture was prepared into granules under a pressure of 0.8 ton in a roller compactor (CHAMUNDA Ltd. CPMRC-200/50 model). After sieving the granules, 0.2 g of sieved magnesium stearate was added thereto and mixed (lubrication step). The resulting granules were tableted with a rectangular punch based on the weight of 275.6 mg per tablet.

### Preparation of Coated Tablet

Using each uncoated tablet of Examples 4 and 5, coated tablet was prepared by the 1^{st} coating of 3.0 mg of Opadry 20A per tablet from Opadry 20A (Colorcone) dispersion in 80% ethanol, and then the 2nd coating of 8.0 mg of Opadry 200F per tablet from Opadry 200F (Colorcone) dispersion in purified water.

### Test Example 3: Dissolution Test for Coated Tablet

For each coated tablet prepared in Examples 4 and 5, a dissolution test was conducted with n=3 under the following conditions according to the paddle method of the 10th revision of the Korean Pharmacopoeia dissolution test method. The results are shown in Table 4 below.

### <Dissolution Test Method>

Dissolution liquid: Purified water
Rotation speed: 50 rpm
Temperature: 37° C.
Timing of collecting dissolution liquid sample: 0.25 hour, 0.5 hour, 0.75 hour, 1 hour, 1.5 hours, 2 hours, 3 hours, 4 hours, 5 hours, 6 hours, 7 hours, 8 hours, 9 hours, 10 hours, 12 hours, 14 hours, 16 hours, 18 hours, 20 hours, 24 hours
Analysis method: HPLC method

TABLE 4

| | Sample | | | | | | | | |
|---|---|---|---|---|---|---|---|---|---|
| | Dissolution rate (%) | | | | | | | | |
| Time (hr) | 0.5 | 1 | 2 | 3 | 5 | 8 | 12 | 16 | 24 |
| Example 4 | 6.1% | 11.9% | 25.0% | 38.3% | 65.8% | 98.2% | 101.2% | 100.9% | 101.4% |
| (coated tablet) | | | | | | | | | |
| Example 5 | 6.3% | 12.6% | 25.9% | 39.5% | 65.1% | 93.8% | 100.5% | 100.4% | 101.1% |
| (coated tablet) | | | | | | | | | |

### Examples 6 to 10

### Preparation of Uncoated Tablet

Tablets were prepared by the same method as Example 1 using the compositions represented in Table 5 below.

TABLE 5

| | Ingredient | Example 6 | | Example 7 | | Example 8 | | Example 9 | | Example 10 | |
|---|---|---|---|---|---|---|---|---|---|---|---|
| | name | Tab.¹ | Prod.² | Tab.¹ | Prod.² | Tab.¹ | Prod.² | Tab.¹ | Prod.² | Tab.¹ | Prod.² |
| Use | (raw product) | (mg) | (g) | (mg) | (g) | (mg) | (g) | (mg) | (g) | (mg) | (g) |
| Active | Ruxolitinib | 26.4 | 2.64 | 26.4 | 2.64 | 26.4 | 2.64 | 26.4 | 2.64 | 26.4 | 2.64 |
| ingredient | phosphate | | | | | | | | | | |
| Sustained-release | Polyox WSR N80 | 75.0 | 7.5 | 75.0 | 7.5 | 40.0 | 4.0 | | | | |
| agent | (M.W.: 20*10⁴) | | | | | | | | | | |
| Sustained-release | Polyox WSR 205 | 75.0 | 7.5 | 75.0 | 7.5 | 130.0 | 13.0 | 85.0 | 8.5 | 35.0 | 3.5 |
| agent | (M.W.: 60*10⁴) | | | | | | | | | | |
| Sustained-release | Polyox WSR N60K | | | | | | | 85.0 | 8.5 | 135.0 | 13.5 |
| agent | (M.W.: 200*10⁴) | | | | | | | | | | |
| Excipient | D-mannitol | 51.2 | 5.12 | | | | | | | | |
| Sustained-release | Macrogol 4000 | | | 70.6 | 7.06 | 50.6 | 5.06 | 50.6 | 5.06 | 50.6 | 5.06 |
| aid | | | | | | | | | | | |
| Binder | Hydroxypropyl | | | 10.0 | 1.0 | 10.0 | 1.0 | 10.0 | 1.0 | 10.0 | 1.0 |
| | cellulose | | | | | | | | | | |
| Sustained-release | Glyceryl | 45.0 | 4.5 | | | | | | | | |
| aid | behenate | | | | | | | | | | |
| Lubricant | Magnesium | 3.0 | 0.3 | 3.0 | 0.3 | 3.0 | 0.3 | 3.0 | 0.3 | 3.0 | 0.3 |
| | stearate | | | | | | | | | | |
| | Total | 275.6 | 27.56 | 260.0 | 26.0 | 260.0 | 26.0 | 260.0 | 26.0 | 260.0 | 26.0 |

¹Amount per tablet
²Amount produced

### Test Example 4: Dissolution Test and Evaluation of Linearity of Dissolution Pattern

For each tablet prepared in Examples 6 to 10, a dissolution test was conducted by the same method as “1. Dissolution test” in Test Example 2, and the results are shown in Table 6 below. The dissolution liquid sample was collected at the timing of initiating the test (0 hour), and 0.25 hour, 0.5 hour, 0.75 hour, 1 hour, 1.5 hours, 2 hours, 3 hours, 4 hours, 5 hours, 6 hours, 7 hours, 8 hours, 9 hours, 10 hours, 12 hours, 14 hours and 16 hours after the test was initiated.

In addition, from the results of the dissolution test, the equation between dissolution time (X) and dissolution rate (Y) was calculated as a trend line through a linear regression model of the dissolution curve from the time zero (0) to the time of 85% dissolution or more, and the results are shown in Table 7 below.

TABLE 6

| Time (hr) | 0 | 0.25 | 0.5 | 0.75 | 1 | 1.5 | 2 | 3 | 4 |
|---|---|---|---|---|---|---|---|---|---|
| Example 6 | 0.0% | 5.7% | 9.5% | 13.2% | 17.0% | 25.9% | 34.9% | 53.4% | 70.8% |
| Example 7 | 0.0% | 5.4% | 9.8% | 14.8% | 20.0% | 30.6% | 44.4% | 73.3% | 89.2%* |
| Example 8 | 0.0% | 4.2% | 7.0% | 9.6% | 12.4% | 18.3% | 25.9% | 41.7% | 59.2% |
| Example 9 | 0.0% | 4.7% | 7.0% | 9.2% | 11.3% | 15.3% | 19.5% | 28.2% | 37.3% |
| Example 10 | 0.0% | 4.3% | 6.2% | 7.9% | 9.7% | 13.0% | 16.3% | 23.3% | 31.4% |

| Time (hr) | 5 | 6 | 7 | 8 | 9 | 10 | 12 | 14 | 16 |
|---|---|---|---|---|---|---|---|---|---|
| Example 6 | 85.3%* | 96.2% | 97.4% | 97.5% | 97.5% | 97.6% | 97.9% | 97.6% | 97.4% |
| Example 7 | 93.3% | 97.4% | 97.1% | 97.2% | 96.9% | 96.7% | 97.8% | 97.1% | 97.5% |
| Example 8 | 74.0% | 85.8%* | 96.3% | 100.0% | 100.3% | 101.0% | 101.6% | 102.4% | 102.6% |
| Example 9 | 46.8% | 58.0% | 66.6% | 73.9% | 80.7% | 86.8%* | 97.7% | 99.0% | 99.1% |
| Example 10 | 39.0% | 46.2% | 53.4% | 60.3% | 67.2% | 73.8% | 85.1%* | 94.9% | 98.4% |

*The timing when the dissolution rate became 85% or more

TABLE 7

| | | Coefficient of |
|---|---|---|
| Sample | Trend line equation | determination, R² |
| Comparative Example 1 | Y = 8.0X + 14.7 | 0.9265 |
| Example 6 | Y = 17.2X + 0.6 | 0.999 |
| Example 7 | Y = 19.2X − 1.2 | 0.9962 |
| Example 8 | Y = 14.7X − 1.3 | 0.9971 |
| Example 9 | Y = 8.8X + 2.3 | 0.9972 |
| Example 10 | Y = 7.1X + 2.4 | 0.9983 |

From the above results, it was confirmed that not only the hydrophobic sustained-release aid but also the hydrophilic sustained-release aid did not affect the zero-order release control, which is a characteristic of Polyox.

### Comparative Example 4: Preparation of Immediate-Release Formulation to be Used as a Comparison Group to Compare the In Vivo Pharmacokinetic Properties to Ruxolitinib Sustained-Release Formulation

26.4 g of Ruxolitinib phosphate, 17.84 g of microcrystalline cellulose, 18.5 g of lactose hydrate, 1.3 g of sodium starch glycolate, 0.8 g of povidone (k-30), and 0.8 g of hydroxypropyl cellulose were sieved and mixed. Then, an appropriate amount of purified water was added thereto, and the resulting mixture was granulized, dried at 50° C. to volatilize the solvent, and then sieved to obtain granules. To the sieved granules, 0.42 g of light anhydrous silicic acid and 0.2 g of magnesium stearate were added and mixed. The mixture was tableted with a rectangular punch based on the weight of 425.0 mg per tablet. The hardness of the uncoated tablet was about 140N.

### Test Example 5: Pharmacokinetic (PK) Test

For each Ruxolitinib tablet (20 mg dose as ruxolitinib) prepared in Comparative Example 4 and Examples 6 and 8 to 10, a nonclinical pharmacokinetic (PK) test was conducted in beagle dogs, in order to compare the in vivo pharmacokinetic properties. The animals for the test were beagle dogs (N=5), which were administered in an empty stomach with water, and blood was collected at predetermined time intervals up to 48 hours. After plasma separation, the blood samples collected for each subject were frozen and stored, and the concentration was analyzed with LC/MS/MS equipment to obtain the concentration in blood over time, and from the data, AUC, Cmax, and half-life were calculated. The results are summarized and shown in Table 8 below.

TABLE 8

| | Cmax | AUC | Half-life |
|---|---|---|---|
| Sample | (ng/mL) | (ng*h/mL) | (hr) |
| Comparative Example 4 | 1114.6 | 3717.6 | 2.25 |
| Example 6 | 654.4 | 2578.3 | 3.34 |
| Example 8 | 727.6 | 3447.6 | 3.42 |
| Example 9 | 507.2 | 3730.5 | 5.55 |
| Example 10 | 509.7 | 3398.1 | 5.39 |

From the above results, it was confirmed that the formulations of Examples showed characteristics of a sustained-release formulation with low Cmax and increased half-life. Particularly, in the case of Examples 8 to 10, it can be seen that the AUC is similar to the AUC of Comparative Example 4 (immediate-release formulation), which is advantageous for drug absorption.

1. An oral tablet formulation comprising:
Ruxolitinib as an active ingredient; and
polyethylene oxide as a sustained-release agent.

2. The oral tablet formulation of claim 1, further comprising a sustained-release aid.

3. The oral tablet formulation of claim 2, wherein the sustained-release aid is a hydrophobic material or a hydrophilic material.

4. The oral tablet formulation of claim 3, wherein the hydrophobic sustained-release aid is selected from glyceryl behenate, ethylcellulose, ammonium methacrylate copolymer, shellac, hydroxypropylmethylcellulose phthalate (HPMC-P), waxes, gums, and combinations thereof.

5. The oral tablet formulation of claim 3, wherein the hydrophilic sustained-release aid is selected from polyethylene glycol, povidone, hydroxypropylcellulose, sugar alcohols, and combinations thereof.

6. The oral tablet formulation of any one of claims 1 to 5, wherein the viscosity average molecular weight of the polyethylene oxide is 100,000 to 4,000,000 g/mol.

7. The oral tablet formulation of any one of claims 1 to 5, wherein the viscosity of the polyethylene oxide is 1000 cP to 10000 cP, based on 1 to 5% (wt/wt) aqueous solution at 25° C.

8. The oral tablet formulation of any one of claims 1 to 5, wherein the amount of the polyethylene oxide is 10 to 90 parts by weight, based on 1 part by weight of Ruxolitinib.

9. The oral tablet formulation of any one of claims 1 to 5, which does not comprise an organic solvent.

10. The oral tablet formulation of any one of claims 1 to 5, further comprising a diluent.

11. The oral tablet formulation of any one of claims 1 to 5, further comprising a lubricant.

12. The oral tablet formulation of any one of claims 1 to 5, further comprising a binder.

13. The oral tablet formulation of any one of claims 1 to 5, further comprising a coating layer.

14. The oral tablet formulation of any one of claims 1 to 5, which exhibits a dissolution pattern with the coefficient of determination, R2 of 0.93 or more obtained by linear regression model of the dissolution time-dissolution rate curve in purified water from the time for the drug dissolution rate being 0% to the time for the drug dissolution rate becoming 85% or more, based on uncoated tablet.

15. A method for preparing an oral tablet formulation, the method comprising:
(1) a step of preparing a mixture comprising Ruxolitinib as an active ingredient and polyethylene oxide as a sustained-release agent; and
(2) a step of tableting the mixture.

16. The method for preparing an oral tablet formulation of claim 15, wherein the mixture tableted in step (2) further comprises a sustained-release aid.

17. The method for preparing an oral tablet formulation of claim 16, wherein the sustained-release aid is a hydrophobic material or a hydrophilic material.

18. The method for preparing an oral tablet formulation of claim 17, wherein the hydrophobic sustained-release aid is selected from glyceryl behenate, ethylcellulose, ammonium methacrylate copolymer, shellac, hydroxypropylmethylcellulose phthalate (HPMC-P), waxes, gums, and combinations thereof.

19. The method for preparing an oral tablet formulation of claim 17, wherein the hydrophilic sustained-release aid is selected from polyethylene glycol, povidone, hydroxypropylcellulose, sugar alcohols, and combinations thereof.

20. The method for preparing an oral tablet formulation of any one of claims 15 to 19, further comprising (3) a step of coating the surface of the tablet (uncoated tablet) obtained as a result of tableting in step (2) with a coating base material.
